(19) United States
(12) Patent Application Publication
(10) Pub. No.: US 2022/0304791 A1
(43) Pub. Date: Sep. 29, 2022

(54) Stepping Doser

(71) Applicant: SHENZHEN INSTITUTES OF ADVANCED TECHNOLOGY CHINESE ACADEMY OF SCIENCES, Shenzhen, Guangdong (CN)

(72) Inventors: Dan LV, Shenzhen, Guangdong (CN); Yang ZHAN, Shenzhen, Guangdong (CN); Yuyao SUN, Shenzhen, Guangdong (CN); Yulin OUYANG, Shenzhen, Guangdong (CN)

(21) Appl. No.: 17/284,761

(22) PCT Filed: Oct. 12, 2019
(86) PCT No.: PCT/CN2019/110869

§ 371 Date: Apr. 12, 2021

(51) Int. Cl. A61D 7/00; A61B 5/291; A61B 5/00

(52) U.S. Cl. CPC A61D 7/00; A61B 5/291; A61B 5/6868; A61B 5/6852; A61B 2503/40

(57) ABSTRACT

The invention provides a stepping doser. The doser comprises a bearing component, a dosing catheter, wiring tubes, a threaded rod, a nut, a signal recorder and a connecting rod; wherein the middle socket of the threaded rod is sheathed with a nut, the lower end of the threaded rod contacts the lower inner wall of the bearing component, the upper end of the threaded rod penetrates the upper part of the bearing component, and the upper end of the threaded rod is provided with a driving structure; The bearing component is also provided with a limit stop for limiting the rotation of the nut so that when the threaded rod rotates, the nut can move up and down; when the nut moves up and down, the dosing catheter can move up and down. The dosing catheter can be stepped with this doser, allowing dosing in a larger area.

### CROSS-REFERENCE TO RELATED APPLICATION

This application is a national stage application of PCT/CN2019/110869. This application claims priority from PCT Application No. PCT/CN2019/110869, filed Oct. 12, 2019, the content of which is incorporated herein in the entirety by reference.

### TECHNICAL FIELD

The invention relates to the field of experimental biomedical devices, especially a stepping doser.

### BACKGROUND

In biological experiments, there are many methods of dosing for experimental animals, such as intraperitoneal injection, intravenous injection and intragastric administration. However, the above methods of dosing is seriously inadequate for the study of neurologic and brain diseases:

First, some drugs cannot penetrate the blood-brain barrier, so the desired effect cannot be reached;

Second, the dose is generally as small as a few microliters or even less.

Traditional methods of dosing for experimental animals are limited for the study of neurologic and brain diseases.

To this end, methods of dosing for nervous centralis have also been developed, which are often used for detecting the response of the target nucleus or brain region to drugs. The current internationally recognized method of acute and chronic microdosing in brains of experimental animals is embedded catheter method: specifically, a tube (catheter) with an inner core with suitable diameter and length is placed in the target brain region or nucleus by positioning with a stereotaxic apparatus, the exposed part of the tube is secured to the skull surface with dental cement and small screws, and signals from neurons need to be detected and recorded after dosing. Changes in the electrical signals of neurons are recorded by dosing for the brain to prove efficacy or observe behavioral changes after dosing.

However, because the catheter is fixed and dosing can only be conducted in a fixed location with this method, dosing for multiple locations with one device and dosing for more comprehensive locations cannot be achieved; moreover, the current method occupies so much volume that a microelectrode array cannot be embedded when there is a catheter in the brain, making it impossible to accurately record electrical signals from neurons after acute dosing.

Therefore, a better doser is needed.

### Contents of the Invention

In order to solve the existing technical defects, the invention provides a stepping doser. The dosing catheter can be stepped with this doser, allowing dosing in a larger area. In addition, in this scheme, a wire electrode for detecting neuronal signals is attached to the dosing catheter, so as to realize the integration of dosing and signal detection. As a result, the dosing area can be detected in a targeted manner, and the signal can be obtained in a more timely and targeted manner, thus making the signal obtained more accurate.

Specifically, the invention provides the following concrete embodiments:

The embodiment of the invention provides a stepping doser. The doser comprises: a bearing component, a dosing catheter connected with the said bearing component, multiple wiring tubes, a threaded rod, a nut fitted with the said threaded rod, a signal recorder for recording neuronal signals and a connecting rod for connecting an external stereotaxic apparatus;

wherein the middle socket of the said threaded rod is sheathed with a nut, the lower end of the said threaded rod contacts the lower inner wall of the said bearing component, the upper end of the said threaded rod penetrates the upper part of the said bearing component, and the upper end of the said threaded rod is provided with a driving structure; The said bearing component is also provided with a limit stop for limiting the rotation of the said nut so that when the said threaded rod rotates, the said nut can move up and down;

the outer wall of the said dosing catheter is fixedly connected with multiple wiring tubes parallelly; one or more the said wiring tubes accommodate wire electrode for detecting and transmitting neuronal signals; the said wire electrode is connected with the said signal recorder; the said nut is fixedly connected with the said dosing catheter, so that when the said nut moves up and down, the said dosing catheter can move up and down.

In a concrete embodiment, the said bearing component comprises the first support plate at the upper end, the second support plate at the lower end and a column; wherein,

the said first support plate and the said second support plate are provided with catheter connecting holes, rod connecting holes and column connecting holes;

the dosing catheter fixedly connected with multiple wiring tubes is connected with the bearing component through the said catheter connecting holes on the said first support plate and the said second support plate respectively; the said connecting rod is fixedly connected with the bearing component through the rod connecting holes on the said first support plate and the said second support plate respectively; the said column is fixedly connected with the said bearing component through the column connecting holes on the said first support plate and the said second support plate respectively;

the said first support plate is also provided with a drive hole for accommodating the said threaded rod; the said second support plate is provided with a groove;

the said nut is arranged between the said first support plate and the said second support plate; the length of the said threaded rod is longer than or equal to the height between the said first support plate and the said second support plate;

the lower end of the threaded rod is sheathed with the said thread through the drive hole, until the lower end of the threaded rod contacts the bottom of the groove.

In a concrete embodiment, there are multiple columns; the number of the said column connecting holes is equal to or greater than the number of the said columns.

In a concrete embodiment, the said bearing component comprises the first support plate at the upper end, the second support plate at the lower end, the third support plate in the middle and a column; wherein,

the said first support plate, the said second support plate and the said third support plate are provided with catheter connecting holes, rod connecting holes and column connecting holes;

The dosing catheter fixedly connected with multiple wiring tubes is connected with the bearing component through the said catheter connecting holes on the said first support plate, the said second support plate and the said third support plate respectively;

the said connecting rod is fixedly connected with the bearing component through the rod connecting holes on the said first support plate, the said second support plate and the said third support plate respectively;

the said column is fixedly connected with the said bearing component through the column connecting holes on the said first support plate, the said second support plate and the said third support plate respectively;

the said first support plate and the said third support plate are also provided with drive holes for accommodating the said threaded rod;

the said nut is arranged between the said first support plate and the said third support plate; the length of the said threaded rod is longer than or equal to the height between the said first support plate and the said second support plate;

the said threaded rod pass through the drive holes on the said first support plate and the said third support plate respectively, the lower end of the said threaded rod is blocked by the said second support plate, and the said nut is sheathed on the said threaded rod in the area between the said first support plate and the said third support plate.

In a concrete embodiment, the lower end of the said threaded rod is also provided with a head; wherein, the area of the cross section of the said head is greater than that of the cross section of the thread of the said threaded rod.

In a concrete embodiment, the said nut is polygonal, the said column is quadrate, and the said limit stop comprises one or more the said columns.

In a concrete embodiment, the said driving structure comprises a cross screw groove or a slotted screw groove.

In a concrete embodiment, the said signal recorder is fixed on the bearing component by bonding; the said dosing catheter fixedly connected with multiple wiring tubes is fixedly connected with the said nut by bonding.

In a concrete embodiment, the said wiring tube is a silicon tube.

In a concrete embodiment, the joint between the said wire electrode and the said signal recorder is encapsulated by applying silver paint.

Therefore, this scheme provides a stepping doser. The dosing catheter can be stepped with this doser, allowing dosing in a larger area. In addition, in this scheme, a wire electrode for detecting neuronal signals is attached to the dosing catheter, so as to realize the integration of dosing and signal detection. As a result, the dosing area can be detected in a targeted manner, and the signal can be obtained in a more timely and targeted manner, thus making the signal obtained more accurate.

### DESCRIPTION OF FIGURES

For a clearer illustration of the technical scheme in this embodiment of the invention, accompanying figures required in the embodiment are briefly introduced below. It should be understood that accompanying figures below are only some embodiments of the invention, so they should not be regarded as limitations of scope. To those of ordinary skills in the art, other related accompanying figures can be obtained on the basis of these figures without creative work.

FIG. 1 is a structure diagram of the stepping doser provided in this embodiment of the invention;

FIG. 2 is a structure diagram of the stepping doser provided in this embodiment of the invention;

FIG. 3 is an explosive view of the stepping doser provided in this embodiment of the invention;

FIG. 4 is a structure diagram of the dosing catheter, wiring tube and wire electrode in the stepping doser provided in this embodiment of the invention;

FIG. 5 is a structure diagram of the threaded rod and nut in the stepping doser provided in this embodiment of the invention;

FIG. 6 is a structure diagram of the signal recorder in the stepping doser provided in this embodiment of the invention.

### LEGEND

1—bearing component; 11—the first support plate; 12—the third support plate; 13—the second support plate; 14—column;
2—dosing catheter; 3—wiring tube;
4—threaded rod; 41—driving structure;
5—nut;
6—signal recorder; 7—connecting rod; 8—wire electrode.

### Specific Implementation Mode

The various embodiments in the disclosure are described more fully below. There may be various embodiments in the disclosure, in which adjustments and changes may be made. However, it should be understood that there is no intention to limit the various embodiments in the disclosure to the particular embodiments in the disclosure, and that the disclosure covers all adjustments, equivalents and/or alternatives in the spirit and scope of the various embodiments involved in the disclosure.

The terms used in the various embodiments in the disclosure are used only to describe the particular embodiments and are not intended to limit the various embodiments in the disclosure. The singular forms of such terms used herein are intended to include their plural forms as well, unless otherwise expressly indicated herein. Unless otherwise defined, all terms used herein (including technical and scientific terms) have the same meanings as those commonly understood by ordinary technicians in the field to which the various embodiments in the disclosure belong. The said terms (such as those defined in dictionaries in general use) will be interpreted as having the same meanings as the contextual meanings in the relevant technical field and will not be interpreted as having idealistic or overly formal meanings, unless otherwise expressly defined in the various embodiments in the disclosure.

### Embodiment 1

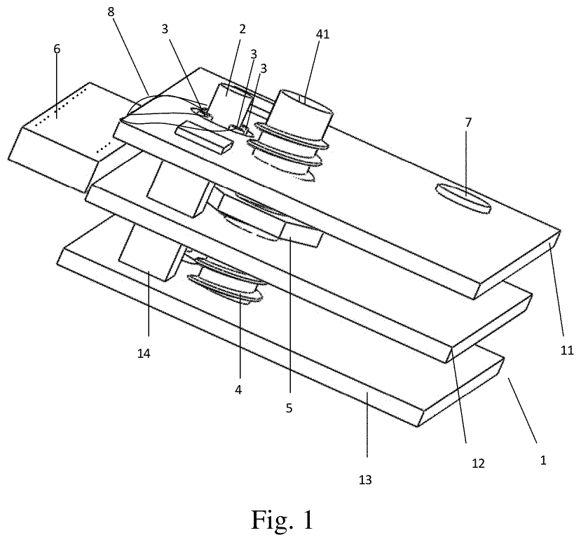

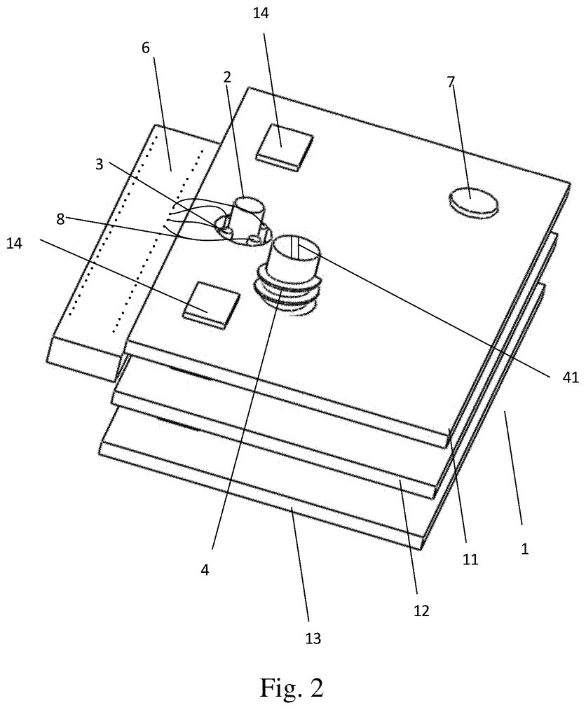

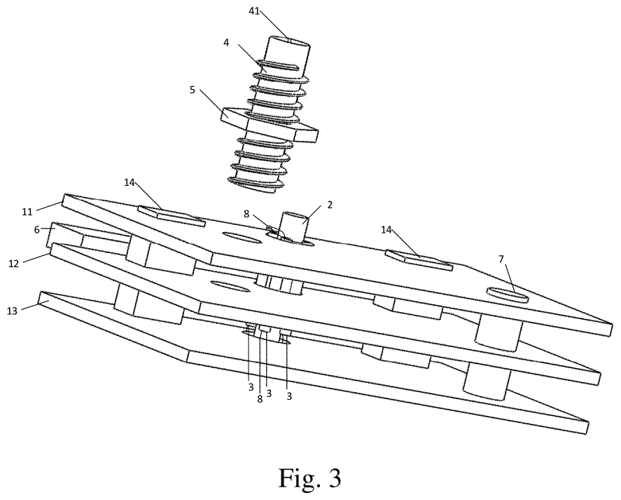

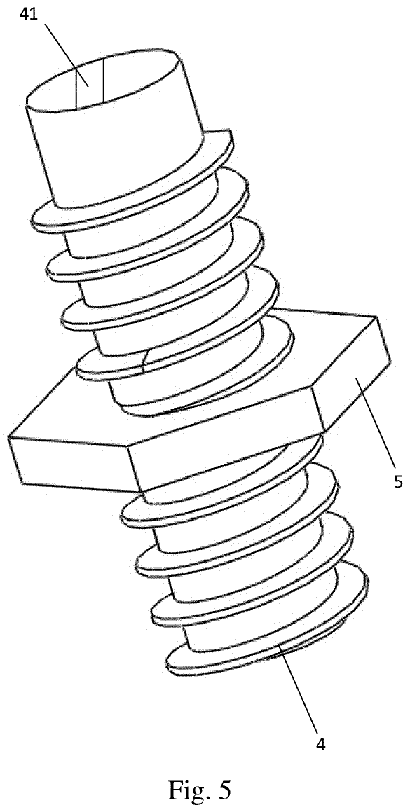

The embodiment of the invention provides a stepping doser. As shown in FIGS. 1-6, the doser comprises: a bearing component 1, a dosing catheter 2 connected with the said bearing component 1, multiple wiring tubes 3, a threaded rod 4, a nut 5 fitted with the said threaded rod 4, a signal recorder 6 for recording neuronal signals and a connecting rod 7 for connecting an external stereotaxic apparatus;

wherein the middle socket of the said threaded rod 4 is sheathed with a nut 5, the lower end of the said threaded rod 4 contacts the lower inner wall of the said bearing component 1, the upper end of the said threaded rod 4 penetrates the upper part of the said bearing component 1, and the upper end of the said threaded rod 4 is provided with a driving structure 41; The said bearing component 1 is also provided with a limit stop for limiting the rotation of the said nut 5, so that when the said threaded rod 4 rotates, the said nut 5 can move up and down;

Specifically, the threaded rod 4 is rotated by the driving structure 41. The threaded rod 4 cannot continue to move downward because its lower end is held up by the inner wall of the lower end of the bearing component 1. As a result, the thread rotates, but the nut 5 is limited by the limit stop, and the threaded rod 4 rotates and drives the nut 5 to move up or down. The dosing catheter 2 is fixedly connected with the nut 5, so moving the nut 5 up and down can drive the dosing catheter 2 to move up and down, thus realizing the stepping of the dosing catheter 2. To this end, the dosing catheter 2 can dose at different positions or different depths under the regulation of the driving structure 41.

There may be different embodiments of concrete limit stops. For example, when the nut 5 is drilled with a circular hole through the upper and lower ends, the limit stop can be a cylindrical steel rod, which limits the nut 5 by passing through the circular hole. In addition, it can be a tubular structure matching the shape of the nut 5, which can also limit the rotation efficiency of the nut 5. Moreover, it can adopt a quadrate column according to the polygonal structure of the periphery of the nut 5. An edge or a surface of the periphery of the quadrate column can resist an edge of the nut 5, which can limit the rotation of the nut 5.

The outer wall of the said dosing catheter 2 is fixedly connected with multiple wiring tubes 3 parallelly; one or more the said wiring tubes 3 accommodate wire electrode 8 for detecting and transmitting neuronal signals; the said wire electrode 8 is connected with the said signal recorder 6; the said nut 5 is fixedly connected with the said dosing catheter 2, so that when the said nut 5 moves up and down, the said dosing catheter 2 can move up and down.

In order to detect neuronal signals at the dosing position in the brain region, the wiring tube 3 is attached to the periphery of the dosing catheter 2, the wiring tube 3 accommodates the wire electrode 8 for signal detection during dosing. Then the signals detected by the wire electrode 8 will be transmitted to the signal recorder 6 for processing, and to other devices for further analysis.

The dosing catheter 2 can be stepped with this doser, allowing dosing in a larger area. In addition, in this scheme, a wire electrode 8 for detecting neuronal signals is attached to the dosing catheter 2, so as to realize the integration of dosing and signal detection. As a result, the dosing area can be detected in a targeted manner, and the signal can be obtained in a more timely and targeted manner, thus making the signal obtained more accurate.

In a concrete embodiment, as shown in FIGS. 1-3, the said bearing component comprises the first support plate 11 at the upper end, the second support plate 13 at the lower end, the third support plate 12 in the middle and a column 14; specially, the column 14 can serve as a limit stop for limiting the rotation of the nut 5, wherein,

the said first support plate 11, the said second support plate 13 and the said third support plate 12 are provided with catheter connecting holes, rod connecting holes and column connecting holes;

The dosing catheter 2 fixedly connected with multiple wiring tubes 3 is connected with the bearing component 1 through the said catheter connecting holes on the said first support plate 11, the said second support plate 13 and the said third support plate 12 respectively;

the said connecting rod 7 is fixedly connected with the bearing component 1 through the rod connecting holes on the said first support plate 11, the said second support plate 13 and the said third support plate 12 respectively;

the said column 14 is fixedly connected with the said bearing component 1 through the column 14 connecting holes on the said first support plate 11, the said second support plate 13 and the said third support plate 12 respectively;

the said first support plate 11 and the said third support plate 12 are also provided with drive holes for accommodating the said threaded rod 4;

the said nut 5 is arranged between the said first support plate 11 and the said third support plate 12; the length of the said threaded rod 4 is longer than or equal to the height between the said first support plate 11 and the said second support plate 13;

the said threaded rod 4 passes through the drive holes on the said first support plate 11 and the said third support plate 12 respectively, the lower end of the said threaded rod 4 is blocked by the said second support plate 13, and the said nut 5 is sheathed on the said threaded rod 4 in the area between the said first support plate 11 and the said third support plate 12.

Specifically, in the embodiment, the bearing component 1 comprises three layers of support plates, which can be metal plates or polymer material plates. Column 14, playing a supporting role, penetrates and fixedly connects the three layers of support plates as a whole. There can be one or more columns 14, as long as the three layers of support plates can be stably supported to provide fixed support for other components.

The threaded rod 4 penetrates the upper support plate and the middle support plate, until the bottom reaches the next layer of the support plate, to prevent the threaded rod 4 to continue to drop. The nut 5 is arranged between the top two layers of support plates; the nut 5 is fixedly connected with the dosing catheter 2;

the dosing catheter 2 fixedly connected with multiple wiring tubes 3 penetrates these three layers of support plates, and moves up and down on the basis of the movement of the nut 5.

The connecting rod 7 is fixed among the three layers of support plates. It can be cylindrical or cubic (i.e. cuboid). The connecting rod 7 is used to connect the external equipment. Specifically, it can be used to connect the stereotaxic apparatus for accurate positioning during the subsequent practical operation. The connecting rod 7 also serves as a support for the three layers of support plates.

In a concrete embodiment, for more stable rotation of the threaded rod 4, the lower end of the said threaded rod 4 is also provided with a head (it can be shaped like the head of a screw or the head of a bolt); wherein, in order to ensure more stable rotation and prevent damage to the lowest support plate, the area of the cross section of the said head is greater than that of the cross section of the thread of the said threaded rod 4.

More specifically, the head can be semicircular or spherical.

Specifically, the said nut 5 is polygonal, such as hexagonal, quadrilateral or pentagonal, etc. The said column 14 is quadrate, and the said limit stop comprises one or more the said columns 14. The column 14 can restrict an edge of the nut 5, thus limiting the rotation of the nut 5 and restricting the moving up and down of the nut 5.

In order to reduce other equipment and avoid occupying more space, the said driving structure 41 comprises a cross screw groove or a slotted screw groove.

To this end, a normal screwdriver or a cross screwdriver can be embedded in the driving structure 41 to manually rotate, thus driving the entire threaded rod 4 to rotate.

In a concrete embodiment, in order to make the connection easier and more reliable, the said signal recorder 6 is fixed on the bearing component 1 by bonding; the said dosing catheter 2 fixedly connected with multiple wiring tubes 3 is fixedly connected with the said nut 5 by bonding.

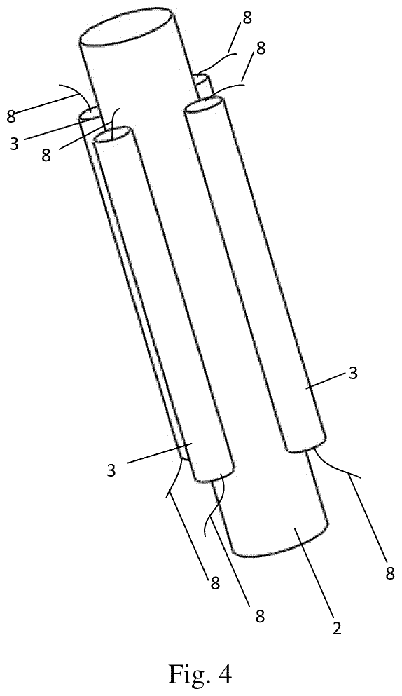

In a concrete embodiment, as shown in FIG. 4, the said wiring tube 3 can be a silicon tube. The silicon tube can be made extremely thin, thus minimizing the space occupation, facilitating the miniaturization of the entire device and minimizing the impact on the dosing area.

In a concrete embodiment, in order to achieve better signal transmission, the joint between the said wire electrode 8 and the said signal recorder 6 is encapsulated by applying silver paint. Silver paint can realize better insulation to ensure the smooth transmission of the signals.

In a concrete embodiment, the length of the dosing catheter 2 can be self-defined according to the brain region, and the length of the wire electrode 8 can be the same as or slightly 1 mm longer than the dosing catheter 2. Then the doser (namely the microelectrode array) is embedded in the brain and cemented with dental cement. The wire electrode 8 is used for recording the electrical signals from the neuron, and the dosing catheter 2 is used for dosing, which allows rapid recording of changes in neurons after dosing.

The concrete bearing component 1 comprises three PCBs (printed circuit boards) of 7.5 mm in length and 6 mm in width. Two copper columns with the length of 0.8 mm and 0.6 mm respectively are fixed on the upper PCB. The middle PCB is also fixed on the two copper columns. The screw (namely an embodiment of the threaded rod 4) is placed between the upper and middle PCBs, and the nut 5 is screwed on under the screw. Then the third PCB is fixed on the longer copper column and pressed against the bottom of the screw (now the head of the screw on the upper PCB can serve as the driving structure 41), so as to prevent the bottom of the screw from moving down, which may make it impossible to step.

The holder (namely the connecting rod 7) is inserted into the three layers of PCBs. The holder is used to fix the whole microelectrode array on the stereotaxic apparatus so that the microelectrode array can be embedded into the target brain region. The joints among the PCBs, the copper columns and the holder are coated with epoxy resin, and a hot air gun is used to blow the epoxy resin until it changes color and solidifies. The epoxy resin shall not be applied to the screw; otherwise, the stepping cannot be realized.

The outer wall of the dosing catheter 2 is coated with AB glue. Nine silicon tubes with a length of 1.2 cm adhere to the outside of the dosing catheter 2, and the silicon tubes encircle the dosing catheter 2. The dosing catheter 2 is inserted into the three layers of PCBs, and the nut 5 and the dosing catheter 2 are glued with AB glue. AB glue shall not be applied to the screw.

After AB glue dries, the connector (namely the signal recorder 6) is glued to the side of the three layers of PCBs. Four strands of the wire electrode 8 are inserted into the silicon tube, the four strands of the wire electrode 8 are connected with the adjacent angles of attack of the signal recorder 6 respectively, then they should be coated with silver paint. The ground electrode reference line can be welded on the preset position of the dosing catheter 2, and the angles of attack of the connector should be sealed with AB glue.

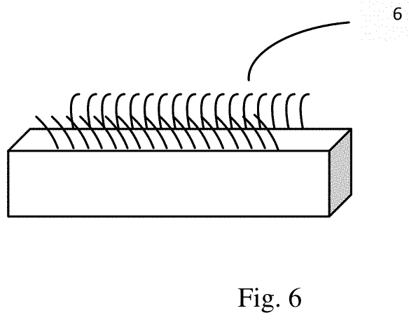

The concrete signal recorder 6 is shown in FIG. 6. It could be an existing device that records electrical signals from neurons, such as, signal recorder 6 with the model of A79026-001, and its DATA CODE can be 1839. Other existing signal recording devices that can record electrical signals from neurons can also be adopted.

Finally, the microelectrode array is placed upside down in the brain of a mouse, and the changes in neuronal signals can be recorded after dosing from the dosing catheter 2.

### Embodiment 2

The embodiment 2 is the same as the embodiment 1, except for the bearing component 1. Specifically, in the embodiment, the said bearing component 1 comprises the first support plate 11 at the upper end, the second support plate 13 at the lower end and a column 14; wherein,

the said first support plate 11 and the said second support plate 13 are provided with catheter connecting holes, rod connecting holes and column connecting holes;

the dosing catheter 2 fixedly connected with multiple wiring tubes 3 is connected with the bearing component 1 through the said catheter connecting holes on the said first support plate 11 and the said second support plate 13 respectively; the said connecting rod 7 is fixedly connected with the bearing component 1 through the rod connecting holes on the said first support plate 11 and the said second support plate 13 respectively; the said column 14 is fixedly connected with the said bearing component 1 through the column connecting holes on the said first support plate 11 and the said second support plate 13 respectively;

the said first support plate 11 is also provided with a drive hole for accommodating the said threaded rod 4; the said second support plate 13 is provided with a groove;

the said nut 5 is arranged between the said first support plate 11 and the said second support plate 13; the length of the said threaded rod 4 is longer than or equal to the height between the said first support plate 11 and the said second support plate 13;

the lower end of the threaded rod 4 is sheathed with the said thread through the drive hole until the lower end of the threaded rod 4 contacts the bottom of the groove.

Specifically, in embodiment 2, there is only two support plates. In order to ensure the stable rotation of the threaded rod 4 and the lower end not to slide or move, a groove is provided to limit the lower end of the threaded rod 4.

In addition, there are multiple columns 14; the number of the said column connecting holes may be equal to or greater than the number of the said columns 14.

Technicians in this field can understand that the drawing is only a schematic diagram of a preferred embodiment, and that the modules or processes in the drawing are not necessarily required for the embodiment of the invention.

Technicians in this field can understand that the modules in the device of the embodiment can be distributed in the device of the embodiment according to the description of the embodiment or can be changed accordingly and distributed in one or more devices that are different from this embodiment. The above embodiment modules can be merged into a module or further split into multiple sub-modules.

The above serial numbers in the invention is for description only and do not represent the advantages and disadvantages of the embodiment.

The embodiments disclosed above are only a few concrete embodiments of the invention. However, the invention is not limited to such embodiments. Any changes that can be contemplated by technicians in this field shall fall under the protection of the invention.

What the claimed is:

1. A stepping doser, wherein the doser comprises: a bearing component, a dosing catheter connected with the said bearing component, multiple wiring tubes, a threaded rod, a nut fitted with the said threaded rod, a signal recorder for recording neuronal signals and a connecting rod for connecting an external stereotaxic apparatus;
wherein the middle socket of the said threaded rod is sheathed with a nut, the lower end of the said threaded rod contacts the lower inner wall of the said bearing component, the upper end of the said threaded rod penetrates the upper part of the said bearing component, and the upper end of the said threaded rod is provided with a driving structure; The said bearing component is also provided with a limit stop for limiting the rotation of the said nut so that when the said threaded rod rotates, the said nut can move up and down;
the outer wall of the said dosing catheter is fixedly connected with multiple wiring tubes parallelly; one or more the said wiring tubes accommodate wire electrode for detecting and transmitting neuronal signals; the said wire electrode is connected with the said signal recorder; the said nut is fixedly connected with the said dosing catheter, so that when the said nut moves up and down, the said dosing catheter can move up and down.

2. The said stepping doser as in claim 1, wherein the said bearing component comprises the first support plate at the upper end, the second support plate at the lower end and a column; wherein,
the said first support plate and the said second support plate are provided with catheter connecting holes, rod connecting holes and column connecting holes;
the dosing catheter fixedly connected with multiple wiring tubes is connected with the bearing component through the said catheter connecting holes on the said first support plate and the said second support plate respectively; the said connecting rod is fixedly connected with the bearing component through the rod connecting holes on the said first support plate and the said second support plate respectively; the said column is fixedly connected with the said bearing component through the column connecting holes on the said first support plate and the said second support plate respectively;
the said first support plate is also provided with a drive hole for accommodating the said threaded rod; the said second support plate is provided with a groove;
the said nut is arranged between the said first support plate and the said second support plate; the length of the said threaded rod is longer than or equal to the height between the said first support plate and the said second support plate;
the lower end of the threaded rod is sheathed with the said thread through the drive hole, until the lower end of the threaded rod contacts the bottom of the groove.

3. The said stepping doser as in claim 2, wherein there are multiple columns; the number of the said column connecting holes is equal to or greater than the number of the said columns.

4. The said stepping doser as in claim 1, wherein the said bearing component comprises the first support plate at the upper end, the second support plate at the lower end, the third support plate in the middle and a column; wherein,
the said first support plate, the said second support plate and the said third support plate are provided with catheter connecting holes, rod connecting holes and column connecting holes;
The dosing catheter fixedly connected with multiple wiring tubes is connected with the bearing component through the said catheter connecting holes on the said first support plate, the said second support plate and the said third support plate respectively;
the said connecting rod is fixedly connected with the bearing component through the rod connecting holes on the said first support plate, the said second support plate and the said third support plate respectively;
the said column is fixedly connected with the said bearing component through the column connecting holes on the said first support plate, the said second support plate and the said third support plate respectively;
the said first support plate and the said third support plate are also provided with drive holes for accommodating the said threaded rod;
the said nut is arranged between the said first support plate and the said third support plate; the length of the said threaded rod is longer than or equal to the height between the said first support plate and the said second support plate;
the said threaded rod pass through the drive holes on the said first support plate and the said third support plate respectively, the lower end of the said threaded rod is blocked by the said second support plate, and the said nut is sheathed on the said threaded rod in the area between the said first support plate and the said third support plate.

5. The said stepping doser as in claim 4, wherein the lower end of the said threaded rod is also provided with a head; wherein, the area of the cross section of the said head is greater than that of the cross section of the thread of the said threaded rod.

6. The said stepping doser as in claim 4, wherein the said nut is polygonal, the said column is quadrate, and the said limit stop comprises one or more the said columns.

7. The said stepping doser as in claim 1, wherein the said driving structure comprises a cross screw groove or a slotted screw groove.

8. The said stepping doser as in claim 1, wherein the said signal recorder is fixed on the bearing component by bonding; the said dosing catheter fixedly connected with multiple wiring tubes is fixedly connected with the said nut by bonding.

9. The said stepping doser as in claim 1, wherein the said wiring tube is a silicon tube.

10. The said stepping doser as in claim 1, wherein the joint between the said wire electrode and the said signal recorder is encapsulated by applying silver paint.
